(19) United States
(12) Patent Application Publication
(10) Pub. No.: US 2024/0350511 A1
(43) Pub. Date: Oct. 24, 2024

(54) TREATMENT OF COVID-19

(71) Applicant: Oxygen Biotech LLC, Wilmington, DE (US)

(72) Inventors: Jan BRABEK, Wilmington, DE (US); Tomas PACAK, Wilmington, DE (US); Milan JAKUBEK, Wilmington, DE (US)

(21) Appl. No.: 18/685,469

(22) PCT Filed: Aug. 22, 2022
(86) PCT No.: PCT/US2022/041079

§ 371 Date: Feb. 21, 2024

Related U.S. Application Data
(60) Provisional application No. 63/235,906, filed on Aug. 23, 2021.

(51) Int. Cl. A61K 31/55; A61K 47/40; A61P 31/14

(52) U.S. Cl. CPC A61K 31/55; A61K 47/40; A61P 31/14

(57) ABSTRACT

Provided herein are compositions useful for the treatment of COVID-19.

### CROSS-REFERENCE

This application claims the benefit of U.S. Provisional Application No. 63/235,906, filed Aug. 23, 2021, which application is incorporated herein by reference in its entirety.

### BACKGROUND

COVID-19 is an infectious disease caused by severe acute respiratory syndrome coronavirus 2 (SARS-CoV-2). Symptoms of COVID-19 include fever, cough, fatigue, breathing difficulties and loss of smell or taste. Some patients develop severe symptoms, including dyspnea, respiratory failure, shock, and multiorgan dysfunction, which can lead to death. Additional treatments for patients with COVID-19 are needed.

### SUMMARY

In certain aspects, disclosed herein is a composition of bazedoxifene or a pharmaceutically acceptable salt thereof and sulfobutyl ether beta-cyclodextrin. In certain aspects, disclosed herein is a liquid pharmaceutical formulation comprising bazedoxifene or a pharmaceutically acceptable salt thereof and sulfobutyl ether beta-cyclodextrin, and further comprising at least one pharmaceutically acceptable solvent. In certain aspects, disclosed herein is a solid pharmaceutical formulation comprising bazedoxifene or a pharmaceutically acceptable salt thereof and sulfobutyl ether beta-cyclodextrin, and further comprising at least one pharmaceutically acceptable excipient. In certain aspects, disclosed herein is an inhalable pharmaceutical formulation comprising bazedoxifene or a pharmaceutically acceptable salt thereof and sulfobutyl ether beta-cyclodextrin, and further comprising at least one pharmaceutically acceptable solvent and at least one pharmaceutically acceptable propellant and/or aerosol-forming gas. In some embodiments, bazedoxifene and sulfobutyl ether beta-cyclodextrin are in a molar ratio of at least about 1:1. In some embodiments, bazedoxifene and sulfobutyl ether beta-cyclodextrin are in a molar ratio of at least about 1:5.

In certain aspects, disclosed herein is a method of treatment of viral infections caused by coronaviruses, comprising the step of administering the composition or the pharmaceutical formulation disclosed herein or a pharmaceutically acceptable salt thereof and sulfobutyl ether beta-cyclodextrin (in a therapeutically effective dose) to a subject in need of such treatment. In some embodiments, the composition is administered by inhalation, intranasally, orally, or intraocularly. In some embodiments, the composition is administered by means of a nebulizer, a nasal spray, an oral formulation, or eye drops. In some embodiments, the composition is administered intravenously.

In certain aspects, disclosed herein is a method of treatment of infections caused by the virus SARS-CoV-2, comprising the step of administering the composition or the pharmaceutical formulation disclosed herein to a subject in need of such treatment. In some embodiments, the composition is administered by inhalation, intranasally, orally, or intraocularly. In some embodiments, the composition is administered by means of a nebulizer, a nasal spray, an oral formulation, or eye drops. In some embodiments, the composition is administered intravenously.

In certain aspects, disclosed herein is a method of treatment of cancer, comprising the step of administering the pharmaceutical formulation disclosed herein (in a therapeutically effective dose) to a subject in need of such treatment. In some embodiments, the pharmaceutical formulation is administered by inhalation, intranasally, orally, or intraocularly. In some embodiments, the pharmaceutical formulation is administered by means of a nebulizer, a nasal spray, a liquid formulation, or eye drops. In some embodiments, the pharmaceutical formulation is administered intravenously. In some embodiments, the cancer is a breast cancer or a pancreatic cancer.

In certain aspects, disclosed herein is a method of treatment of osteoporosis, comprising the step of administering the pharmaceutical composition disclosed herein (in a therapeutically effective dose) to a subject in need of such treatment. In some embodiments, the osteoporosis is postmenopausal osteoporosis.

In certain aspects, disclosed herein is a salt of bazedoxifene with sulfobutyl ether beta-cyclodextrin.

In certain aspects, disclosed herein is a composition for use as a medicament. In certain aspects, disclosed herein is a composition for use in the treatment of a disease selected from viral infections caused by coronaviruses, SARS-CoV-2 infection, cancer and osteoporosis.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 depicts solubility isotherms for Bazedoxifene acetate with selected cyclodextrins, interpolation fitted lines.

FIG. 2 depicts solubility isotherms for Bazedoxifene acetate with selected cyclodextrins, interpolation fitted linear lines.

FIG. 3 depicts solubility isotherms for Bazedoxifene acetate with selected cyclodextrins, polynomic interpolation fitted lines.

FIG. 4 depicts UV-Vis spectra, showing the aggregation of bazedoxifene acetate in saline.

FIG. 5 depicts UV-Vis spectrum of HPBCD encapsulate of bazedoxifene acetate, diluted 1000×.

FIG. 6 depicts UV-Vis spectrum of gamma-CD encapsulate of bazedoxifene acetate, diluted 1000×.

FIG. 7 depicts UV-Vis spectrum of SBECD encapsulate of bazedoxifene acetate, diluted 1000×.

FIG. 8 depicts UV-Vis spectrum of beta-CD encapsulate of bazedoxifene acetate, diluted 1000×.

FIG. 9 depicts powder X-ray diffraction patterns of BAZ (top line), SBECD (middle line) and BAZ:SBECD complex (bottom line)

FIG. 10 depicts Differential Scanning Calorimetry thermograms of BAZ (middle line), SBECD (top line) and BAZ:SBECD complex (bottom line)

FIG. 11 depicts the structure of BAZ and atom numbering used for evaluation of NMR spectra.

FIG. 12 depicts ¹H NMR spectrum of BAZ with integrations and full assignment (600 MHZ, 298 K, MeOD: D₂O 3:4 (v/v)).

FIG. 13 depicts DEPT and HSQC spectrum of BAZ with full assignment.

FIG. 14 depicts the structure of SBECD and atom numbering used for evaluation of NMR spectra.

FIG. 15 depicts ¹H NMR spectrum of BAZ:SBECD complex with full assignment (600 MHZ, 298 K, D₂O).

FIG. 16 depicts DEPT and HSQC spectrum of BAZ:SBECD complex with full assignment (600 MHZ, 298 K, D₂O).

FIG. 17 depicts the structure of BAZ:SBECD complex, based on NMR study.

FIG. 18 depicts powder X-ray diffraction patterns of BAZ (top line), BAZ:GCD complex (middle line) and GCD (bottom line)

FIG. 19 depicts Differential Scanning Calorimetry thermograms of BAZ (top line), GCD (middle line) and BAZ:GCD complex (bottom line)

FIG. 20 depicts the structure and atom numbering of GCD.

FIG. 21 depicts ¹H NMR spectrum of BAZ:GCD 1:1 with partial assignment (600 MHz, 298 K, D₂O).

FIG. 22 depicts the expanded ¹H spectrum of BAZ:GCD 1:1 showing the A) aromatic region of BAZ and B) the core region of GCD.

FIG. 23 depicts the structures of BAZ:GCD 1:1.

FIG. 24 depicts ¹H NMR spectrum of BAZ:GCD complex with partial assignment (600 MHZ, 298, D₂O: DMSO-d₆ 6:1 (v/v)).

FIG. 25 depicts the expanded ¹H spectrum of BAZ:GCD complex showing the A) aromatic region of BAZ and B) the core region of GCD with assignment of the peaks (600 MHZ, 298, D₂O: DMSO-d₆ 6:1 (v/v)).

FIG. 26 depicts the mean plasma concentration-time profiles for Cohort B (linear scale).

### DETAILED DESCRIPTION

Bazedoxifene belongs to a group of medicinal products designated as selective estrogen receptor modulators (SERM). Bazedoxifene shows affinity to estrogen receptors but manifests tissue-selective estrogenic effects and is used for the prevention of osteoporosis in menopausal women and for the treatment of medium to severe vasomotor response symptoms related to menopause (e.g., hot flashes, sweating). Bazedoxifene has estrogen-agonist effects on bones and the cardiovascular system, but estrogen-antagonist effects on the tissue of breast and uterus. This dissimilar activity is of key importance in order to attain the beneficial effects of estrogens on bones when reducing resorption and attaining the turning point, but also to eliminate the potentially harmful effects of the estrogen-induced stimulation of breast and uterine tissues. For example, bazedoxifene has been studied for the purpose of treatment of oncological diseases, such as breast cancer, colon cancer, pancreas cancer, stomach cancer and endometrium cancer

There are several serious difficulties preventing the utilization of bazedoxifene, such as the low solubility of the medicinal product (0.000564 mg/mL; drugbank.ca) in aqueous systems, and the resulting low bioactivity. There is still a need to develop solubilization systems for bazedoxifene and its derivatives and analogues which could be used in pharmaceutical formulations to enable the use of bazedoxifene and its derivatives and analogues in the treatment of COVID-19 disease by targeted delivery to the lungs and brain.

### Compounds

In certain aspects, described herein are pharmaceutical compositions of compounds of bazedoxifene or pharmaceutically acceptable salts thereof which have a significantly increased solubility compared to the compound of bazedoxifene or pharmaceutically acceptable salts thereof, and which exhibit an improved biological efficiency.

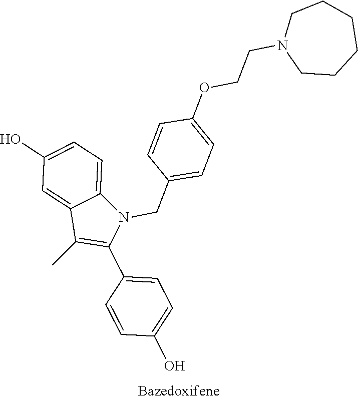 [SMILES: Cc1c(-c2ccc(O)cc2)n(Cc2ccc(OCCN3CCCCCC3)cc2)c2ccc(O)cc12]

In a first aspect of the invention, a pharmaceutical composition of a compound of general bazedoxifene; and sulfobutyl ether beta-cyclodextrin;

is provided.

### Pharmaceutical Compositions

Without being limited by theory, in certain aspects, the interaction of bazedoxifene or a pharmaceutically acceptable salt thereof with a cyclodextrin significantly differs depending on the type of the cyclodextrin. The interaction with hydroxypropyl-beta-cyclodextrin for dissolving bazedoxifene or its salts has been shown to be unsuitable for pharmaceutical uses, due to formation of poorly bioavailable aggregates which are known to decrease biological efficiency and result in poorly reproducible quality in pharmaceutical products. Beta-cyclodextrin forms a complex with the compound of bazedoxifene or a pharmaceutically acceptable salt thereof which is unstable. Alpha-cyclodextrins have a cavity which is too small to include the compound of bazedoxifene or a pharmaceutically acceptable salt thereof. Use of substituted gamma-cyclodextrins did not increase the solubility of compounds of bazedoxifene or pharmaceutically acceptable salts thereof in a sufficient manner. Therefore, the use of cyclodextrins for improving the solubility of compounds of bazedoxifene or pharmaceutically acceptable salts thereof did not seem to lead to compositions having the desired properties for pharmaceutical use. Sulfobutyl ether beta-cyclodextrin (SBECD) exhibited the desired properties for practical use in pharmaceutical formulations, which may include significantly increased solubility without decreasing biological activity due to the undesirable formation of aggregates.

In some embodiments, sulfobutyl ether beta-cyclodextrin forms a stable salt with bazedoxifene. In some embodiments, the sulfo groups of sulfobutyl ether beta-cyclodextrin form salt with the OH and O groups of bazedoxifene.

In some embodiments, the compositions described herein form an encapsulate/salt for bazedoxifene or its salt with sulfobutyl ether beta-cyclodextrin.

In some embodiments, the molar ratio of bazedoxifene or its salt to sulfobutyl ether beta-cyclodextrin in the pharmaceutical formulation is within the range of 1:1 to 1:20, 1:1 to 1:15, 1:1 to 1:10, 1:1 to 1:9, 1:1 to 1:8, 1:1 to 1:7, 1:1 to 1:6, 1:1 to 1:5, 1:1 to 1:4, 1:1 to 1:3, or 1:1 to 1:2. In some embodiments, the molar ratio of bazedoxifene or its salt to sulfobutyl ether beta-cyclodextrin is about 1:1, about 1:2, about 1:3, about 1:4, about 1:5, about 1:6, about 1:7, about 1:8, about 1:9, about 1:10, about 1:11, about 1:12, about 1:13, about 1:14, about 1:15, about 1:16, about 1:17, about 1:18, about 1:19 or about 1:20.

In some embodiments, the concentration of bazedoxifene or its salt is at least about 1 mg/mL, about 2 mg/mL, about 3 mg/mL, about 4 mg/mL, about 5 mg/mL, about 6 mg/mL, about 7 mg/mL, about 8 mg/mL, about 9 mg/mL, about 10 mg/mL, about 12.5 mg/mL, about 15 mg/mL, about 17.5 mg/mL, about 20 mg/mL, or more than about 20 mg/mL. In some embodiments, the concentration of bazedoxifene or its salt is no more than about 1 mg/mL, about 2 mg/mL, about 3 mg/mL, about 4 mg/mL, about 5 mg/mL, about 6 mg/mL, about 7 mg/mL, about 8 mg/mL, about 9 mg/mL, about 10 mg/mL, about 12.5 mg/mL, about 15 mg/mL, about 17.5 mg/mL, about 20 mg/mL, or about 20 mg/mL.

Pharmaceutically acceptable salts of bazedoxifene are salts with pharmaceutically acceptable acids. The pharmaceutically acceptable salts may include hydrochloride, chloride, hydrobromide, bromide, hydroiodide, iodide, nitrate, phosphate, monohydrogenphophate, dihydrogenphosphate, phosphonate, sulfate, hydrogensulfate, sulfite, acetate, phenylacetate, trifluoroacetate, acrylate, ascorbate, benzoate, chlorobenzoate, methylbenzoate, methoxybenzoate, dinitrobenzoate, hydroxybenzoate, acetoxybenzoate, naphthalene-2-benzoate, isobutyrate, phenylbutyrate, hydroxybutyrate, butyn-1,4-dioate, hexyn-1,6-dioate, caprate, caprylate, cinnamate, citrate, formiate, fumarate, glycolate, heptanoate, hippurate, lactate, malate, maleate, hydroxymaleate, malonate, mandelate, mesylate, nicotinate, isonicotinate, oxalate, phthalate, terephthalate, propionate, phenylpropionate, salicylate, sebacate, suberate, sulfonate, benzenesulfonate, p-bromophenylsulfonate, chlorobenzenesulfonate, ethanesulfonate, 2-hydroxyethanesulfonate, methanesulfonate, p-toluenesulfonate, naphthalene-1-sulfonate, naphthalene-2-sulfonate, xylenesulfonate, tartarate.

In some embodiments, the pharmaceutically acceptable salt of bazedoxifene is bazedoxifene acetate.

In some embodiments, the sulfobutyl ether beta-cyclodextrin is preferably a beta-cyclodextrin substituted with 6-7 (preferably 6.5) sulfobutyl ether groups. In some embodiments, the sulfobutyl ether beta-cyclodextrin may be a beta-cyclodextrin substituted with about 1, 2, 3, 4, 5, 6, 7, or more sulfobutyl ether groups. In some embodiments, the sulfobutyl ether groups may be appended onto any position of the beta-cyclodextrin core. In some embodiments, the countercation of the sulfo group is an alkali metal. In some embodiments, the counteraction of the sulfo group is a sodium or potassium, or an ammonium cation.

In some embodiments, the pharmaceutical composition of the present invention is prepared by combining a solution of the corresponding cyclodextrin with a solution or suspension of bazedoxifene or a pharmaceutically acceptable salt thereof.

In some embodiments, the pharmaceutical composition of the present invention is prepared by combining a solution of the corresponding cyclodextrin with bazedoxifene or a pharmaceutically acceptable salt thereof in solid form. In some embodiments, sonication is applied in order to speed up and complete the dissolution of bazedoxifene or its salt.

In some embodiments, the pharmaceutical composition of the present invention is prepared by mechanical treatment (mechanical processing) of a mixture of bazedoxifene or a pharmaceutically acceptable salt thereof in solid form with the corresponding cyclodextrin in solid form. In some embodiments, the mechanical treatment includes milling or grinding of the mixture. In some embodiments, the mechanical treatment includes milling or grinding for at least 600 rpm. In some embodiments, the mechanical treatment includes milling for at least 10 minutes.

In some embodiments, the pharmaceutical compositions of the invention may be formulated into solid or liquid pharmaceutical formulations.

Liquid pharmaceutical formulations may include, for example, solutions, solutions for injections, solutions for infusions, solutions for inhalation and sprayable solutions. The liquid pharmaceutical formulations include the pharmaceutical composition containing bazedoxifene and sulfobutyl ether beta-cyclodextrin, and at least one pharmaceutically acceptable solvent, optionally also at least one pharmaceutically acceptable excipient.

Pharmaceutically acceptable solvents may preferably include water, saline, phosphate buffered saline, and pharmaceutically acceptable buffers.

Solid pharmaceutical formulations may include, for example, tablets, dragees, hard capsules, soft capsules, implantable formulations, ointments, gels, suppositories and the like. The solid pharmaceutical formulations include the pharmaceutical composition containing bazedoxifene or its salt and sulfobutyl ether beta-cyclodextrin, and at least one pharmaceutically acceptable excipient.

Pharmaceutically acceptable excipients include anti-adherents, binders, coatings, colorants, disintegrants, fillers, flavors, glidants, lubricants, preservatives, sorbents, sweeteners, vehicles. Suitable excipients are known to a person skilled in the art, and can be selected by the skilled person based on the specific formulation and intended use. Lists of suitable excipients are available in pharmacopoeias and databases, such as https://www.accessdata.fda.gov/scripts/cder/iig/index.cfm.

In some embodiments, the pharmaceutical formulations are formulations for inhalation or for intranasal administration.

Pharmaceutically acceptable propellant gases commonly used in pharmaceutical aerosols include chlorofluorocarbons, fluorocarbons (e.g., trichloromonofluoromethane, dichlorodifluoromethane), hydrocarbons (e.g., propane, butane, isobutane), hydrochlorofluorocarbons and hydrofluorocarbons, inert gases (e.g., nitrogen, NO₂, CO₂), air, and oxygen. In some embodiments, the propellant gases are kept in the dosing device under a pressure which is higher than atmospheric pressure.

In some embodiments, the pharmaceutically acceptable aerosol-forming gases includes air or inert gases such as nitrogen.

In some embodiments, the compositions described herein are administered by nebulizer. A nebulized solution is one dispersed in air to form an aerosol, and a nebulizer generates very fine liquid droplets suitable for inhalation into the lung. Nebulizers typically use compressed air, ultrasonic waves, or a vibrating mesh to create a mist of the droplets and may also have a baffle to remove larger droplets from the mist by impaction. A variety of nebulizers are available for this purpose, such as ultrasonic nebulizers, jet nebulizers and breath-actuated nebulizers. In use, mouthpieces or masks are typically attached to a patient to aid delivery of the nebulized solution.

In some embodiments, the compositions described herein are delivered intranasally. Intranasal administration of compounds offers several advantages over traditional surgical, intravenous or oral routes for administration across the blood brain barrier (BBB). Intranasal administration to the olfactory region avoids gastrointestinal destruction and hepatic first pass metabolism, such as destruction of drugs by liver enzymes, allowing more drug to be cost-effectively, rapidly, and predictably bioavailable than if it were administered orally. Intranasal administration can provide ease, convenience and safety. Intranasal drug administration is generally painless (taking into consideration that pain may be a subjective measurement which varies by patient) and does not require sterile technique, intravenous catheters or other invasive devices, and is generally immediately and readily available for all patients. Intranasal administration can rapidly achieve therapeutic brain and spinal cord drug concentrations.

In some embodiments, the compositions described herein are delivered intravenously.

In some embodiments, the composition of the invention will enable to develop liquid or oral formulations with an improved bioavailability, allowing to decrease the necessary doses of the active ingredient and to decrease the amount of ballast substances to be taken with the active ingredient.

### Methods of Treatment

In some embodiments, the composition comprising bazedoxifene or a pharmaceutically acceptable salt thereof and sulfobutyl ether beta-cyclodextrin is suitable for therapeutic or preventative use. In some embodiments, the compositions described herein are suitable for use in treatment of viral diseases. In some embodiments, the compositions described herein are suitable for use in treatment of diseases caused by coronaviruses. In some embodiments, the compositions described herein are suitable for use in treatment of infections caused by the virus SARS-CoV-19 or viral strains derived therefrom, i.e., in treatment of disease COVID-19.

In some embodiments, bazedoxifene and its analogues prevent the cytokine storm which is a common complication of the COVID-19 disease. In some embodiments, bazedoxifene and its analogues prevent ARDS related to COVID-19. In some embodiments, the effect is due to its anti-inflammatory activity and anti-IL6 signaling. In some embodiments, bazedoxifene exhibits a direct antiviral activity against SARS-CoV-2.

In some embodiments, inhalatory or intranasal administration of a pharmaceutical composition described herein is suitable for the treatment of COVID-19 because it allows to deliver the active ingredient directly to the infected tissues (very typically, lung or brain and central nervous system). In some embodiments, intravenous administration of a pharmaceutical composition described herein is suitable for the treatment of COVID-19.

In some embodiments, the compositions described herein are useful for the treatment of cancers or osteoporosis, in line with the known therapeutic activities of bazedoxifene. In some embodiments, the compositions described herein is useful for the treatment of breast cancer and pancreatic cancer. In some embodiments, the compositions described herein is useful for the treatment of osteoporosis. In some embodiments, the composition is formulation to be delivered intraocularly. In some embodiments, the composition is delivered as eye drops.

### Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a”, “an” and “the” include plural references unless the context clearly dictates otherwise. For example, the term “a sample” includes a plurality of samples, including mixtures thereof.

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative or quantitative and qualitative determinations. Assessing can be relative or absolute. “Detecting the presence of” can include determining the amount of something present in addition to determining whether it is present or absent depending on the context.

The terms “subject,” “individual,” or “patient” are often used interchangeably herein. A “subject” can be a biological entity containing expressed genetic materials. The biological entity can be a plant, animal, or microorganism, including, for example, bacteria, viruses, fungi, and protozoa. The subject can be tissues, cells and their progeny of a biological entity obtained in vivo or cultured in vitro. The subject can be a mammal. The mammal can be a human. The subject may be diagnosed or suspected of being at high risk for a disease. In some cases, the subject is not necessarily diagnosed or suspected of being at high risk for the disease.

The term “in vitro” is used to describe an event that takes places contained in a container for holding laboratory reagent such that it is separated from the biological source from which the material is obtained. In vitro assays can encompass cell-based assays in which living or dead cells are employed. In vitro assays can also encompass a cell-free assay in which no intact cells are employed.

As used herein, the term “about” a number refers to that number plus or minus 10% of that number. The term “about” a range refers to that range minus 10% of its lowest value and plus 10% of its greatest value.

As used herein, the terms “treatment” or “treating” are used in reference to a pharmaceutical or other intervention regimen for obtaining beneficial or desired results in the recipient. Beneficial or desired results include but are not limited to a therapeutic benefit and/or a prophylactic benefit. A therapeutic benefit may refer to eradication or amelioration of symptoms or of an underlying disorder being treated. Also, a therapeutic benefit can be achieved with the eradication or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

The section headings used herein are for organizational purposes only and are not to be construed as limiting the subject matter described.

### EXAMPLES

The following examples are included for illustrative purposes only and are not intended to limit the scope of the invention.

### Example 1: Materials and Methods

Phase solubility studies: Isothermal solubility assays were performed according to Higuchi-Connors in purified water at room temperature, wherein CD solutions of discrete concentrations were weighed, and an excess amount of bazedoxifene acetate was added (see also Advances in Analytical Chemistry and Instrumentation ed. C. N. Reilly, Whiley, New York, 1965, vol. 4, pp. 117-212.). After 24 hours of equilibration time at 25±3° C. (using a magnetic stirrer at 500 rpm), the dissolved bazedoxifene concentrations were determined by HPLC after filtration through a syringe filter having a polyethylene sulfone membrane of 0.45 μm nominal pore size.

X-ray powder diffraction: Powder X-ray diffraction patterns were recorded with a X'pert Pro MDP (PANalytical B.v., The Netherlands) X-ray diffractometer using Cu K-α radiation and a Ni metal filter.

Differential Scanning Calorimetry: The measurements were performed using a Modulated DSC 2920 device (TA Instruments, DE, USA). The samples (1-5 mg) were measured in sealed Al pans at a heating rate of 10 K/min. For temperature and enthalpy calibration of the DSC instrument a pure In metal standard was applied.

NMR: All ¹H and 2D NMR experiments were performed at 298 K on a 600 MHz Varian DDR NMR spectrometer equipped with a 5 mm inverse-detection gradient (IDPFG) probe. Standard pulse sequences and processing routines available in VnmrJ 4.1 were used. ¹H chemical shifts (δ) were referenced to the residual HOD peak (δ=4.7900 ppm) present in D₂O. ¹H spectra were recorded from 16 scans with 2 s relaxation delay applied.

2D Rotating Frame Overhauser Enhancement Spectroscopy (ROESY) spectra were recorded from 8 scans/increment using 2 s relaxation delay and 512 increment, the spinlock time was set to 300 ms.

Distortionless enhancement by polarization transfer edited heteronuclear single quantum coherence (DEPT and HSQC) spectra were recorded from 4 scans/increment using 256 increments and Is relaxation delay.

### Example 2: Phase Solubility Studies

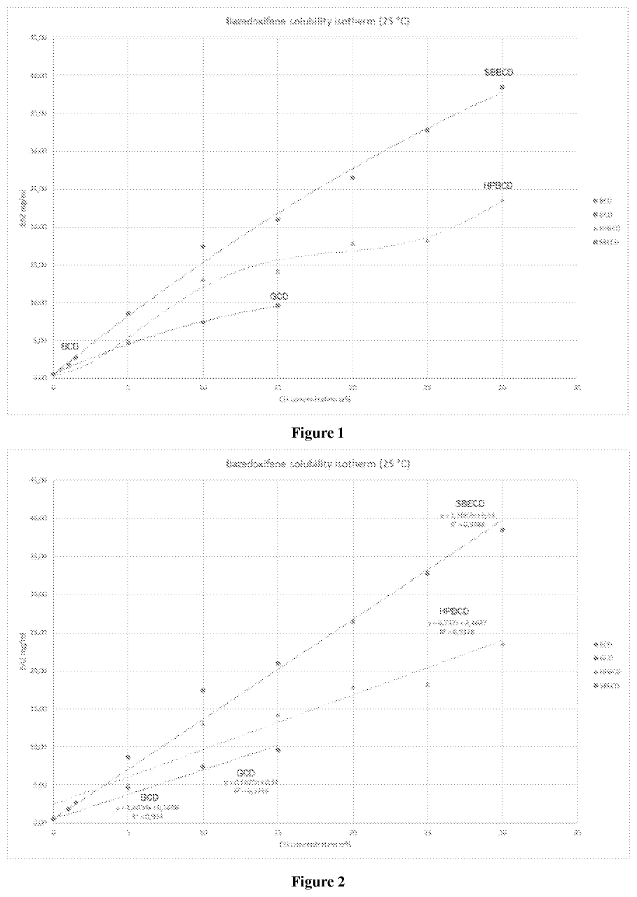

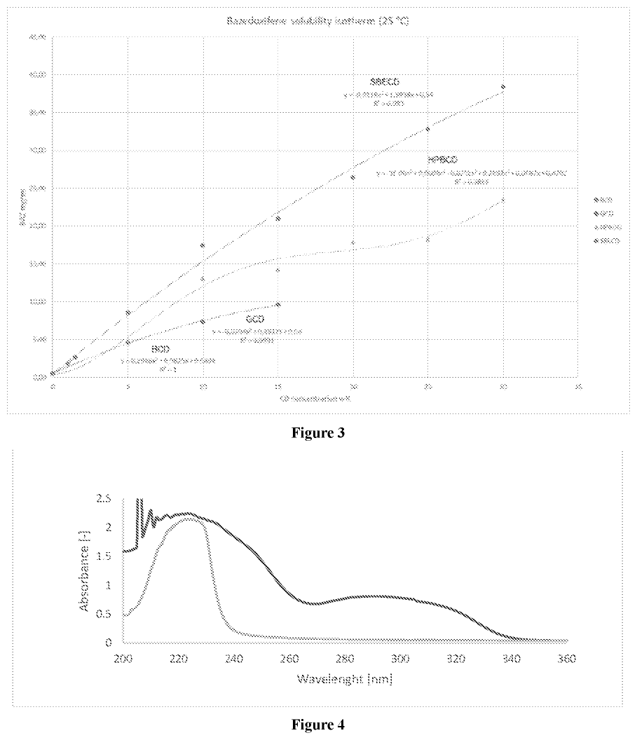

Solubility of compositions of bazedoxifene acetate with various cyclodextrins was explored. FIG. 1 shows the most relevant results obtained for compositions with beta-cyclodextrin (BCD), gamma-cyclodextrin (GCD), hydroxypropyl-beta-cyclodextrin (HPBCD) and sulfobutyl ether beta-cyclodextrin (SBECD) with fitted interpolation lines. FIG. 2 shows the same data, with linear interpolation fits. Linear interpolation fit data are summarized in Table 1. FIG. 3 shows the same data with polynomic interpolation fits, the polynomic interpolation fit data are summarized in Table 2. BAZ is an abbreviation for bazedoxifene acetate.

TABLE 1
Linear interpolation fit data for solubility
isotherm shown in FIG. 2.

| | | | Poly- | Conc. |
|---|---|---|---|---|
| Encapsulation | | | nomial | Range CD |
| systems | Linearity | R2 | level | (% w/w) |
| BAZ:BCD | Y = 1.4034x + 0.5088 | 0.9940 | 2 | 0-2.68 |
| BAZ:GCD | Y = 0.6425x + 0.54 | 0.9795 | 2 | 0-15 |
| BAZ:HPBCD | Y = 0.717x + 2.4627 | 0.9378 | 2 | 0-30 |
| BAZ:SBECD | Y = 1.3087x + 0.54 | 0.9888 | 2 | 0-30 |

TABLE 2
Polynomic interpolation fit data for
solubility isotherm shown in FIG. 3.

| | | | Poly- | Conc. |
|---|---|---|---|---|
| Encapsulation | | | nomial | Range CD |
| systems | Linearity | R2 | level | (% w/w) |
| BAZ:BCD | Y = 0.2946x2 + | 0.9999 | 2 | 0-2.68 |
| | 0.9825x + 0.5404 | | | |
| BAZ:GCD | Y = −0.0194x2 + | 0.9991 | 2 | 0-15 |
| | 0.8917x + 0.54 | | | |
| BAZ:HPBCD | Polynomial | 0.9881 | >4 | 0-30 |
| BAZ:SBECD | Y = −0.0118x2 + | 0.9930 | 2 | 0-30 |
| | 1.5938x + 0.54 | | | |

The results for the tested cyclodextrins showed that a sufficient concentration of BCD was not achieved due to its poor solubility in aqueous media. HPBCD resulted in a composition to which the solubility isotherm corresponded to a polynomial of fourth order, which indicated the formation of undesirable aggregates that negatively interfere with the solubilization and dispersion of bazedoxifene acetate, or any of its cyclodextrin complexes which may have formed. The solubility isotherms obtained using GCD and SBECD allowed for a close-to-linear fit, which indicated that there was minimal, if any, formation of aggregates.

The aggregating behavior of HPBCD alone was observed by some authors in the academic literature, e.g. Sá Couto A R, Ryzhakov A, Loftsson T. Materials (Basel). 2018; 11(10): 1971, doi: 10.3390/ma11101971. The observed aggregates of HPBCD were measured to be in the size range of 80-800 nm.

The undesired aggregating behavior of the HPBCD encapsulate implied that as the concentration of cyclodextrins increased, and the proportion of cyclodextrin units that did not complex or dissolve the drug may have also increased, the additional cyclodextrin units may have participated in the aggregation of already formed cyclodextrin-drug complexes. This phenomenon indicated that using HPBCD to formulate a dosage form may result in the formation and release of a mixture of different aggregates with significantly different pharmacokinetic properties.

Further experiments were carried out to study the behavior of the bazedoxifene-cyclodextrin compositions.

Due to the poor solubility of bazedoxifene in aqueous media, DMSO was used for preparing its stock solutions as part of the standard procedure in biological studies. In view of the above, a bazedoxifene acetate drug stock solution (4.8 mg/mL) was prepared and a proportional portion was diluted with saline (11.2 μL to 1000 μL). The drug concentration was 10 mmol/mL. The sample was left for 4 hours at room temperature. UV-visible absorbance spectra of the solution before and after filtration (employing a 0.22 μm disc filter) were measured. After filtration, there was a sharp decrease in absorbance features corresponding to the drug (FIG. 4), suggesting the formation of undesirable aggregates that were removed by filtration. This suggests that the limited efficacy of this drug, observed in its medical use, could be due to its strong aggregation.

To verify the hypothesis that compositions of HPBCD and bazedoxifene exhibited an increased aggregation, while those of GCD or SBECD with bazedoxifene did not show this undesirable aggregation behavior, the solubility and aggregation of bazedoxifene cyclodextrin encapsulates were studied. 80 mg of bazedoxifene acetate and 600 mg of cyclodextrin (selected from BCD, HPBCD, GCD and SBECD) were weighed into a 15 mL tube and 5.4 mL of distilled water was added. The formulations were placed in an ultrasonic bath at elevated temperature (40° C.) for 6 hours. After cooling to room temperature, UV-Vis spectra were measured for unfiltered solutions as well as for the solutions after filtration using a 0.22 μm disc filter.

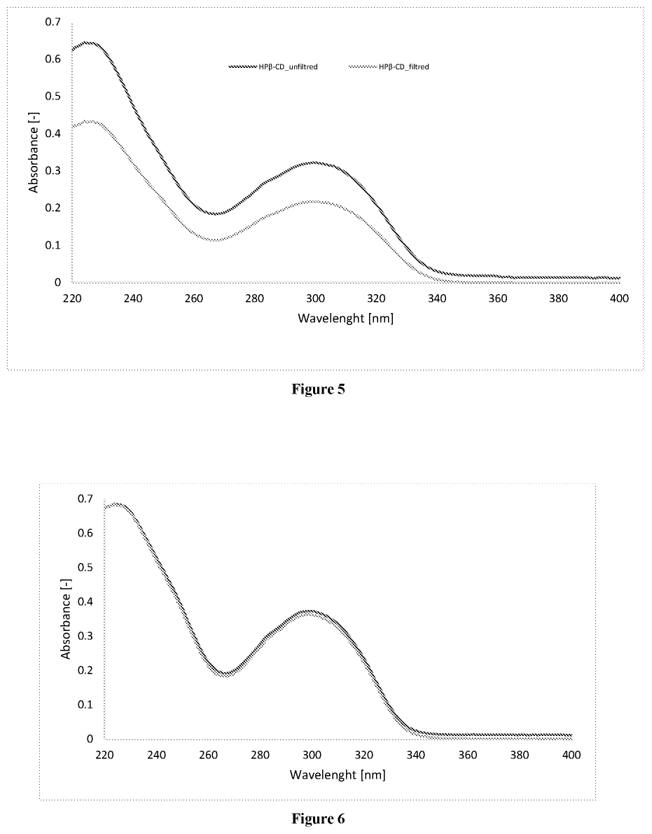

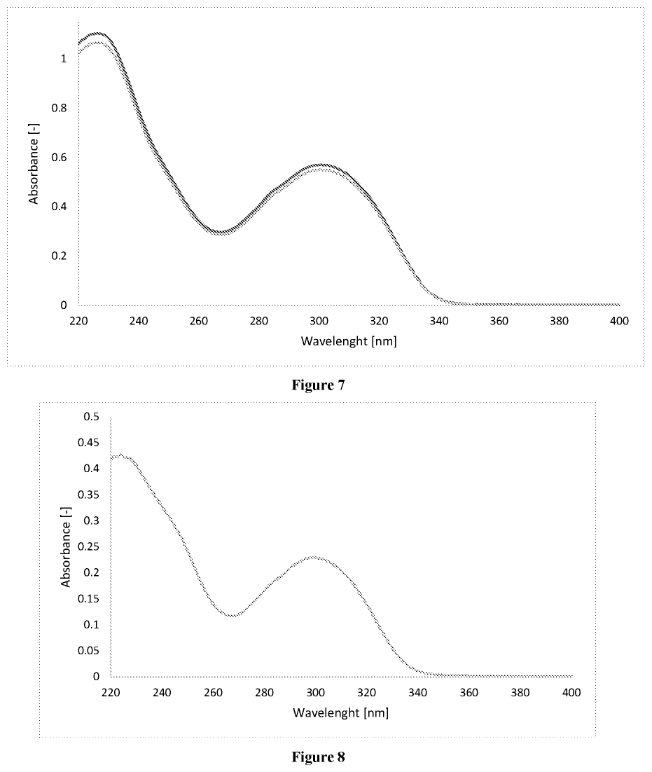

The bazedoxifene acetate encapsulate with HPBCD (FIG. 5) was observed to have a lower throughput through the filter used than was observed with the encapsulates of BAZ with GCD (FIG. 6) and SBECD (FIG. 7). In the case of BCD, only UV-visible absorbance data from the filtered solution were measured because the unfiltered solution formed a slurry (FIG. 8).

The study of the solubility of bazedoxifene acetate using binding isotherms showed that the GCD composition of bazedoxifene had lower solubility than the HPBCD composition; however, it also revealed the strongly undesirable aggregation of HPBCD complexes of bazedoxifene in biological systems. These aggregates may be sufficiently large such that the effect of the drug is significantly reduced, due to a decrease in the amount of drug that may absorbed by the cells, or possibly its lifetime in the blood prior to interception by macrophages (Hoshyar N, Gray S, Han H, Bao G. The effect of nanoparticle size on in vivo pharmacokinetics and cellular interaction. Nanomedicine (Lond). 2016; 11 (6): 673-692. DOI: 10.2217/nnm.16.5). In contrast, a suitably selected cyclodextrin formulation could have a significantly lower particle size and thus a significantly greater potency. Therefore, it could be concluded that the biological efficacy of the bazedoxifene composition with HPBCD may be significantly lower than that of compositions with GCD or SBECD.

### Example 3: Solubility of Bazedoxifene Acetate in Gamma-Cyclodextrin in Water

11.9 mg of bazedoxifene acetate was weighed into a 1.5 mL microtube. 1.2 mL of a 40 mg/mL solution of GCD in distilled water was added into the microtube. The solution was sonicated at 40° C. for 6 hours. After dissolution of bazedoxifene acetate, aliquots of the solution were diluted at 1:10 and 1:100 ratios with a solution containing 40 mg/mL GCD in distilled water. Thus, the resulting three solutions had the bazedoxifene concentrations: 9.9 mg/mL, 0.99 mg/mL and 0.099 mg/mL. The concentration of GCD remained the same in all solutions, specifically 40 mg/mL.

Aliquots of the solutions were filtered through a 0.22 μm disc filter. By comparing the unfiltered and filtered solutions by absorbance measurements at 300 nm (a characteristic maximum absorbance wavelength for bazedoxifene), it was found that 97% of bazedoxifene acetate was dissolved.

### Example 4: Solubility of Bazedoxifene Acetate in Gamma-Cyclodextrin in Saline

6 mg of bazedoxifene acetate was weighed into a 1.5 mL microtube. 1.5 mL of a 40 mg/mL solution of gamma-cyclodextrin in saline was added into the microtube. The solution was sonicated at 40° C. for 6 hours. After dissolution of bazedoxifene acetate, aliquots of the solution were diluted at 1:10 and 1:100 ratios with a solution of 40 mg/mL GCD in saline. Thus, the resulting three solutions had the bazedoxifene concentrations: 4 mg/mL, 0.4 mg/mL and 0.04 mg/mL. The concentration of gamma-cyclodextrin remained the same in all solutions, specifically 40 mg/mL.

Aliquots of the solutions were filtered through a 0.22 μm disc filter. By comparing the unfiltered and filtered solutions by absorbance measurement at 300 nm, it was found that 92% of bazedoxifene acetate was dissolved.

### Example 5: Solubility of Bazedoxifene Acetate in Gamma-Cyclodextrin in Buffer Solution

3.2 mg of bazedoxifene acetate was weighed into a 1.5 mL microtube. 1.2 mL of a 40 mg/mL solution of GCD in phosphate-buffered saline was added into the microtube. The solution was sonicated at 40° C. for 6 hours. After dissolution of bazedoxifene acetate, aliquots of the solution were diluted at 1:10 and 1:100 ratios with a solution of 40 mg/mL GCD in phosphate-buffered saline. Thus, the resulting three solutions had the bazedoxifene concentrations: 9.9 mg/mL, 0.99 mg/mL and 0.099 mg/mL. The concentration of GCD remained the same in all solutions, specifically. 40 mg/ml.

Aliquots of the solutions were filtered through a 0.22 μm disc filter. By comparing the unfiltered and filtered solutions by absorbance measurement at 300 nm, it was found that 95% of bazedoxifene acetate was dissolved.

### Example 6: Solubility in Water of Bazedoxifene Acetate in Gamma-Cyclodextrin Produced by Grinding

50 mg of bazedoxifene acetate, 250 mg of GCD and 10 g of grinding balls were weighed into a 100 mL steel grinding vessel. The resulting mixture was ground for 20 minutes at a set grinding speed of 1000 rpm and a grinding temperature of 37° C.

20 mg of the ground mixture was weighed into a microtube and combined with 0.5 mL distilled water. The sample was then sonicated for 5 minutes at 45° C.

An aliquot of the sonicated solution was filtered through a 0.22 μm disc filter. By comparing the unfiltered and filtered solutions by absorbance measurement at 300 nm, it was found that 99% of the starting bazedoxifene was dissolved.

### Example 7: Study on Interaction Between Bazedoxifene Acetate and Cyclodextrins (CDs)

NMR experiments were used to study solubility and the interaction between bazedoxifene acetate (BAZ) and cyclodextrins (CD). The assignment of BAZ was done by using 2D NMR and ¹³C NMR experiments. The sample preparation was done by dissolving 5 mg of BAZ in 700 μL deuterated solvent. The compositions of the solutions that were prepared are listed in Table 3. In case of BAZ:SBECD 1:1, the mixture was filtered before it was transferred to the NMR tube. All the samples were measured using standard 5 mm glass NMR sample tubes.

A feasibility study was conducted on the suitability of widely (especially orally) applicable cyclodextrins for encapsulation of bazedoxifene acetate (BAZ): Sulfobutylether beta-cyclodextrin (SBECD) and gamma-cyclodextrin (GCD) were selected as the best performing cyclodextrins for BAZ solubilization.

BAZ:SBECD complex was prepared as follows: Calculated on dry basis, 11.0 g SBECD (equivalent with 11.76 g actual weighing due to 6.5 w % water content) was dissolved in 44 mL Millipore Synergy purified water. 0.90 g BAZ was dissolved in the medium. This SBECD/BAZ ratio ensured an approximate 30% excess of SBECD as compared to the equilibrium ratio, which was within the industrially applicable range and was found applicable in earlier experiences developing compositions of cyclodextrins with other active pharmaceutical agents. Instead of the usual 2 hours stirring at room temperature (by magnetic stirrer at 500 rpm), the mixture only became a clear solution after being stirred overnight because the BAZ used was not in a micronized form. After a clarification filtration through a 0.45 μm nominal pore size PES membrane, the solution was frozen, lyophilized, and then ground to a fine powder form.

The BAZ:SBECD 1:1 (mol/mol) composition was prepared by combining equimolar amounts of solid bazedoxifene acetate and solid SBECD in the deuterated solvent or solvent mixture specified in Table 3. The resulting solutions were filtered before being transferred to the NMR tube. All the samples were measured using standard 5 mm glass NMR sample tubes.

TABLE 3
Compositions of the samples prepared for the experimental
investigations (SBECD = sulfobutyl ether beta-cyclodextrin

| Composition of the sample | Compounds | Quantity | |
|---|---|---|---|
| BAZ | BAZ | 5.15 | mg |
| | MeOD | 300 | μL |
| | D₂O | 400 | μL |
| BAZ:SBECD (isolated | BAZ:SBECD complex | 71.03 | mg |
| complex) | D₂O | 700 | μL |
| BAZ:SBECD from dry | BAZ | 5.00 | mg |
| solids (1:1 mol/mol) | SBECD | 22.75 | mg |
| | MeOD | 200 | μL |
| | D₂O | 700 | μL |

BAZ:GCD complex was prepared as follows: Calculated on dry basis, 19.2 g GCD (equivalent with 21.3 g actual weighing due to 10.0 w % water content) and 0.78 g BAZ were weighed into a porcelain mortar. The powder mixture was homogenized in dry form, then wetted with 10 mL purified water. The obtained paste was kneaded for 15 minutes (over this time, 2×1.0 mL portions of additional purified water were added to enable kneading) until the consistency of the substance prevented further kneading (hardening was observed). The GCD: BAZ ratio utilized ensured an approximate 30% excess of GCD compared to the equilibrium ratio, which was within the industrially applied quantity range and was found was found applicable in earlier experiences developing compositions of cyclodextrins with other active pharmaceutical agents. The wet paste was dried over P₂O₅ in vacuo for 48 hours, and then ground to a fine powder form.

The BAZ:GCD 1:1 (mol/mol) composition was prepared by combining equimolar amounts of solid bazedoxifene acetate and solid GCD in the deuterated solvent mixture specified in Table 4. The dissolved mixture was filtered before being transferred to the NMR tube. All the samples were measured using standard 5 mm glass NMR sample tubes.

TABLE 4
Compositions of the samples prepared for the experimental
investigations (GCD = gamma-cyclodextrin)

| Label of the sample | Compounds | Quantity | |
|---|---|---|---|
| BAZ | BAZ | 5.15 | mg |
| | MeOD | 300 | μL |
| | D₂O | 400 | μL |
| BAZ:GCD from dry solids | BAZ | 5.44 | mg |
| (1:1 mol/mol) | GCD | 13.60 | mg |
| | D₂O | 1 | mL |
| BAZ:GCD complex | BAZ:GCD complex | 30.00 | mg |
| | DMSO-d₆ | 100 | μL |
| | D₂O | 600 | μL |

### Example 8: BAZ:SBCD Instrumentation Analyses

### X-Ray Powder Diffraction of BAZ:SBECD

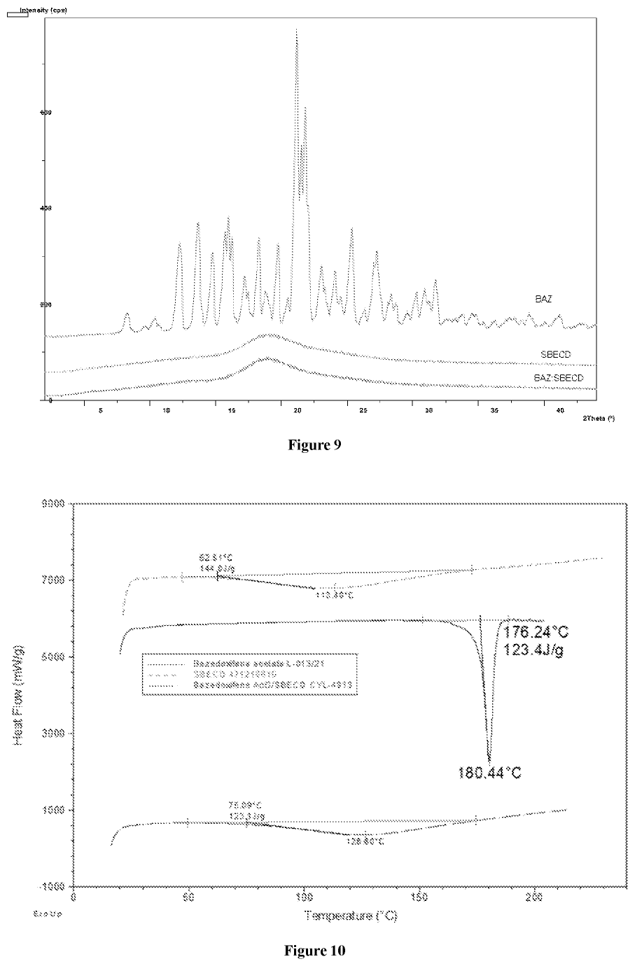

Powder X-ray diffraction (PXRD) patterns of the starting materials and the resulting binary BAZ:SBECD composition were recorded and are shown in FIG. 9. The PXRD data indicated that while the starting material drug substance BAZ was a crystalline substance, on the contrary, SBECD was amorphous. The PXRD profile of the BAZ:SBECD composition also indicated an amorphous structure, suggesting that BAZ was in molecularly dispersed state within the cyclodextrin matrix and that a complex was formed between the two constituents.

### Differential Scanning Calorimetry of BAZ:SBECD

Differential Scanning Calorimetry (DSC): DSC thermograms of the starting materials and the resulting binary BAZ:SBECD composition were recorded (FIG. 10). The thermal analysis showed that crystalline BAZ had melting enthalpy peak at 180.4° C. Amorphous SBECD had no such characteristic phase transition in the studied temperature range, only a wide endothermic effect was observed due to loss of water. The DSC profile of the BAZ:SBECD composition also indicated an amorphous structure, suggesting—similarly to the results of PXRD analysis—that crystalline BAZ was absent in the sample. These results provided further evidence that a complex was formed between the two constituents.

### Nuclear Magnetic Resonance (NMR) Studies of BAZ:SBECD

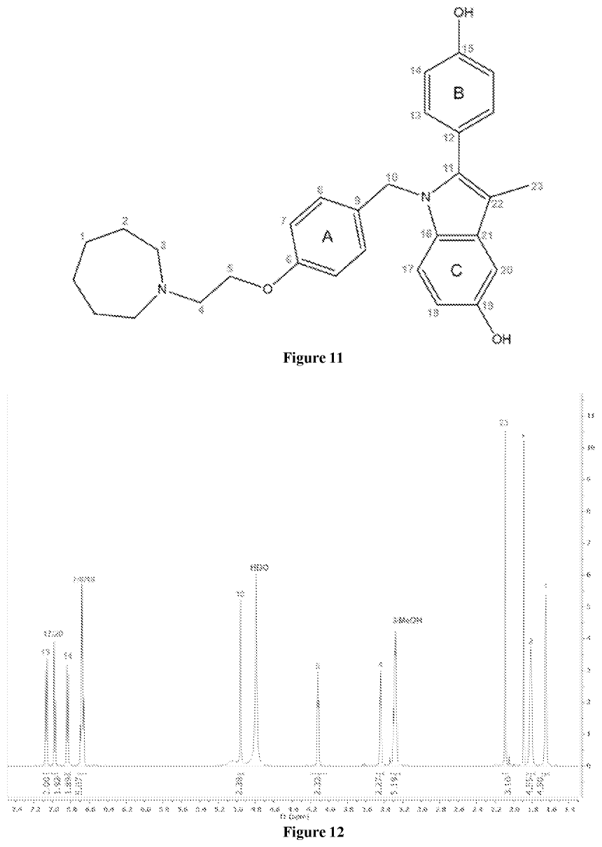

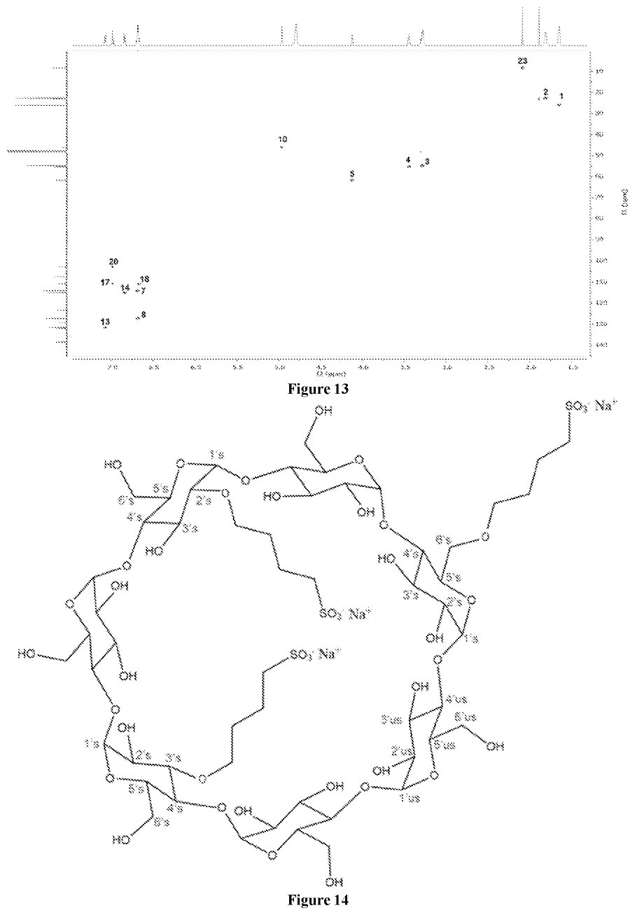

The NMR spectral assignment of BAZ was done by employing ¹H NMR spectroscopy in combination with 2D NMR and ¹³C NMR experiments. The structure and atom numbering scheme of BAZ referred to herein is shown in FIG. 11. FIG. 12 shows the ¹H NMR spectrum of BAZ with integrations and full assignment. FIG. 13 shows a DEPT and HSQC spectrum of BAZ with full assignment. To map the intramolecular interactions of BAZ, a 2D ROESY experiment was run using the same set of parameters as were used for the investigation of the complexes, such that the observed cross peaks could be easily distinguished from those resulting from host-guest type interactions.

As SBECD was not a single-isomeric compound but was rather a randomly substituted derivative of BCD, a complex mixture of isomers with different degrees of substitution (DS) and substitution patterns was present. Therefore, the assignment of NMR spectra collected from samples containing SBECD could be challenging due to the presence of overlapping signals. In the case of SBECD, this isomeric heterogeneity was further complicated by the fact that the proton NMR resonances of some part of the sulfobutyl side chains resonated at the same frequency as do those corresponding to the core region (comprising the signals of the glucopyranose units besides the anomeric protons) of the CD unit. Consequently, the determination of the site of the interactions at the atomic level was impossible by using routine NMR spectroscopy methods in conjunction with the materials employed. The structure and atom numbering scheme of SBECD referred to herein is shown in FIG. 14.

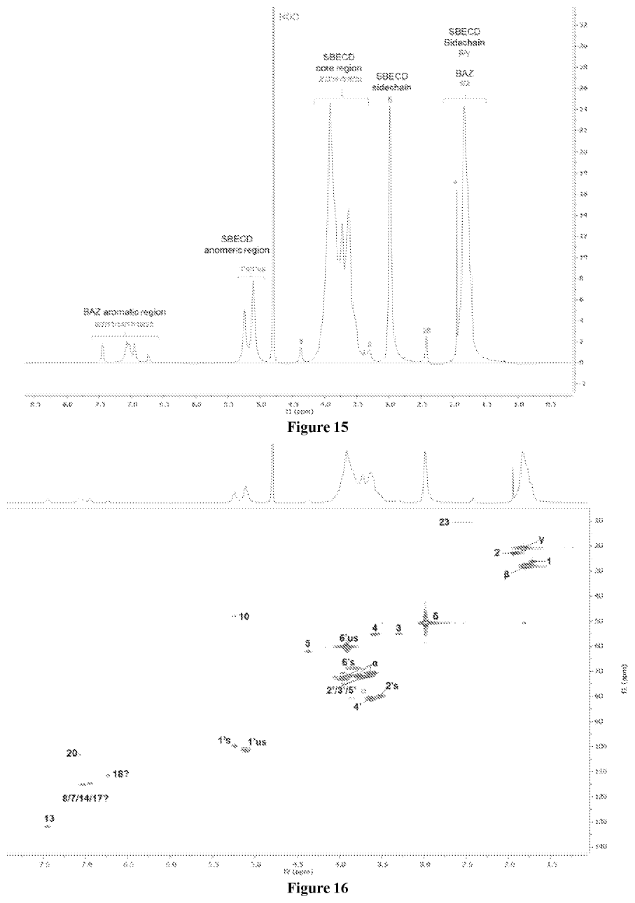

FIG. 15 shows the ¹H NMR spectrum of the BAZ:SBECD complex with full assignment. In the high frequency region of the spectrum (δ_{1H}=6.5-7.5 ppm) the aromatic protons of BAZ (7, 8, 13, 14, 17, 18, 20) can be found. It is easy to recognize that these signals were remarkably broadened in comparison to those observed in the spectrum of pure BAZ, which indicated some interaction with SBECD in solution. Between δ_{1H}=5.0-5.5 ppm the anomeric protons of the CD unit were generally observed. In the case of randomly substituted CD derivatives, the NMR signals corresponding to two types of anomers were separated, the substituted type (1's), corresponding to glucopyranose units that bore a side chain at least in one position, and the unsubstituted type (1'us), representing glucopyranose units that lacked side chains. This duplication of the signals could be characteristically observed for the rest of the signals of the glucose unit as well (which could be seen in the DEPT and HSQC spectrum depicted in FIG. 16). Signals corresponding to the core region of the CD were routinely observed between δ_{1H}=3.5-4.3 ppm, which included all the protons of the glucose unit (2′, 3′, 4′, 5′, and 6′) except those at the anomeric site (1 and 1′). Signals corresponding to the side chain protons of SBECD closest to the CD cavity (α) were also observed in the same region. While the signals corresponding to the middle methylene units of the sidechain (β and γ) were observed between δ_{1H}=1.5-2.0 ppm, completely overlapping with signals of the azepane moiety (1 and 2) of BAZ, the terminal side chain methylene protons adjacent to the sulfo-group (δ) resonated around δ_{1H}=3.0 ppm in a separated region. Further separated resonance signals of BAZ (5, 3 and 23) could be identified at δ_{1H}=4.4, δ_{1H}=3.3 and δ_{1H}=2.4, respectively. The peak at δ_{1H}=4.79 ppm was the HDO signal from the solvent.

The DEPT and HSQC spectrum of BAZ:SBECD complex with the ¹H spectrum on the x-axis is shown in FIG. 16 with full assignment.

Comparing with the DEPT and HSQC spectrum of BAZ, the number of distinguishable signals in spectrum of BAZ:SBECD had clearly decreased. It was important to note, that while in the case of BAZ alone, the signals corresponding to the seven chemically-inequivalent sets of aromatic protons (7, 8, 13, 14, 17, 18, and 20) were separated, in the case of BAZ:SBECD complex only five separated cross-peaks in the same region of the spectrum could be identified. Based on the ¹³C chemical shift values, only two of the signals (13 and 20) could be assigned with confidence, as a remarkable ¹³C chemical shift change generally did not occur due to complexation or solvent change. On the contrary, it could be seen that an aromatic proton of BAZ with higher δ_{13C} (probably 8) shifted upfield (towards lower frequencies) and was overlapping with other peaks. (It was also important to emphasize, that the difference in the solvent composition of the sample intrinsically influences the observed resonances).

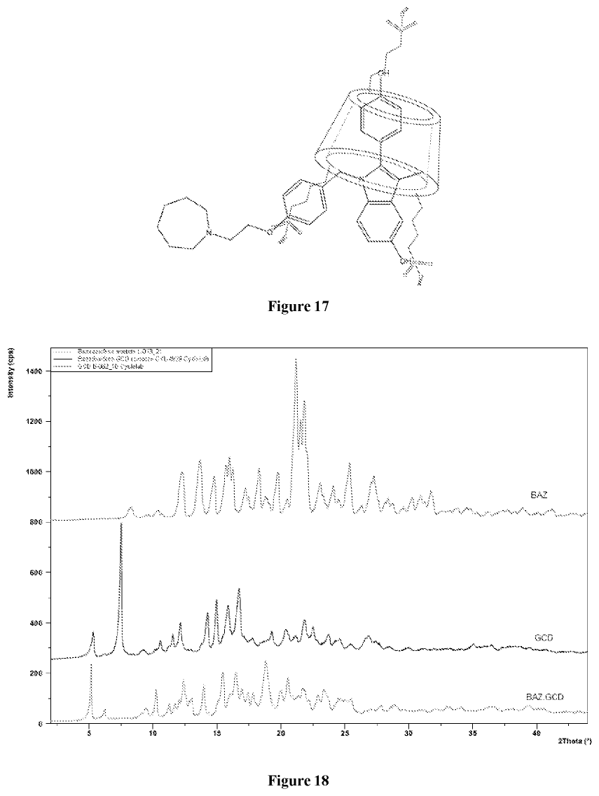

Summarizing the above listed findings, it could be concluded that BAZ was able to form a host-guest complex with SBECD. A plausible structure for this complex, based on 2D ROESY analysis, was composed as shown in FIG. 17.

### Example 9: BAZ:GCD Instrumentation Analyses

### X-Ray Powder Diffraction of BAZ:GCD

Powder X-ray diffraction (PXRD) patterns of the starting materials and the resulting binary BAZ:GCD composition were recorded and are shown in FIG. 18. The PXRD data indicates that the starting material drug substance BAZ and GCD are both crystalline substances. The profile of the BAZ:GCD composition is also crystalline, though PXRD profile is significantly different as compared to that of either starting material, suggesting that a complex was formed between the two constituents.

### Differential Scanning Calorimetry of BAZ:GCD

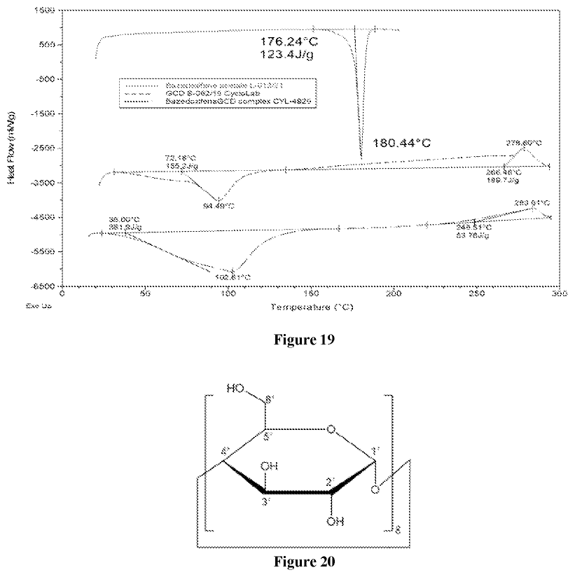

Differential Scanning Calorimetry (DSC) thermograms of the starting materials and the resulting binary BAZ:GCD composition were recorded (FIG. 19). The thermal analysis showed that crystalline BAZ has melting enthalpy peak at 180.4° C. GCD has no such characteristic phase transition in the studied temperature range: a wide endothermic effect is observed due to loss of water around 100° C., and a feature suggestive of thermal decomposition is observed at temperatures over ca. 250° C. The DSC profile of the BAZ:GCD composition suggests that BAZ is in a molecularly dispersed state in the GCD matrix, as no crystalline BAZ is observed in the sample. Crucially, the observation that crystalline BAZ is absent in the binary composition provides further evidence that a complex was formed between the two constituents.

### Nuclear Magnetic Resonance (NMR) Studies of BAZ:GCD

The NMR spectral assignment of BAZ was done by employing ¹H NMR spectroscopy in combination with 2D NMR and ¹³C NMR experiments. The structure and atom numbering scheme of BAZ referred to herein is shown in FIG. 11. FIGS. 12 and 13 show the ¹H- and DEPT and HSQC spectra of BAZ with full assignment. The structure and numbering scheme of GCD referred to herein is shown in FIG. 20.

In this experiment, equimolar amounts of BAZ and GCD were weighed together and dissolved in D₂O. FIG. 21 shows the ¹H NMR spectrum of the BAZ:GCD 1:1 with partial assignment.

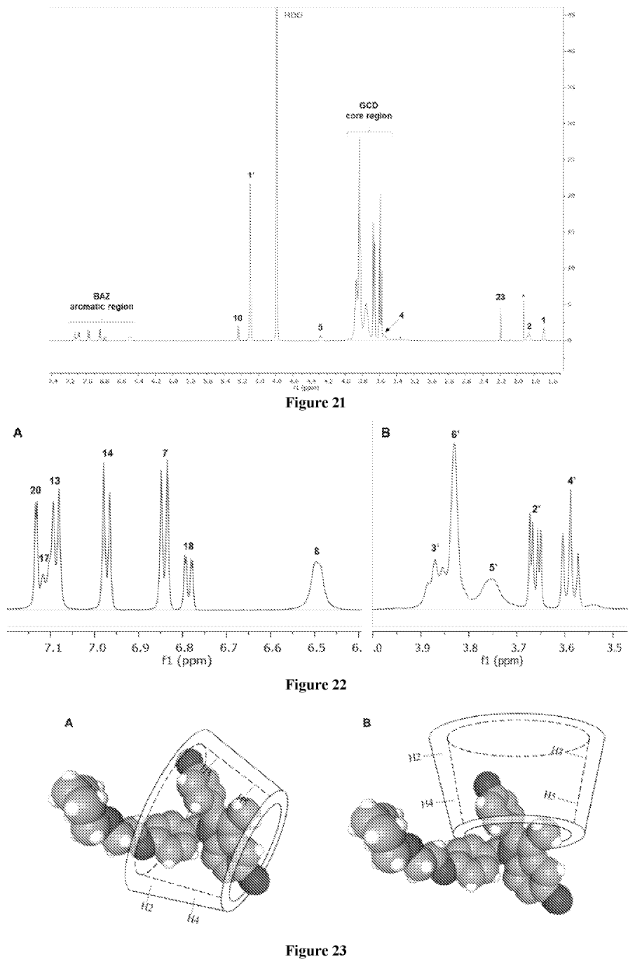

Signals corresponding to the core region of native CDs generally appeared in between δ_{1H}=3.5-4.0 ppm, comprising all the protons of the glucopyranose rings besides those at the anomeric positions. Signals corresponding to protons bound to the anomeric carbon atoms were observed between δ_{1H}=5.0-5.2 ppm, in a separated region of the spectrum. Another well separated set of signals in the high frequency region of the ¹H NMR spectrum corresponded to the aromatic region of BAZ. The assignment of the two above mentioned sets of signals was determined as shown in FIG. 22.

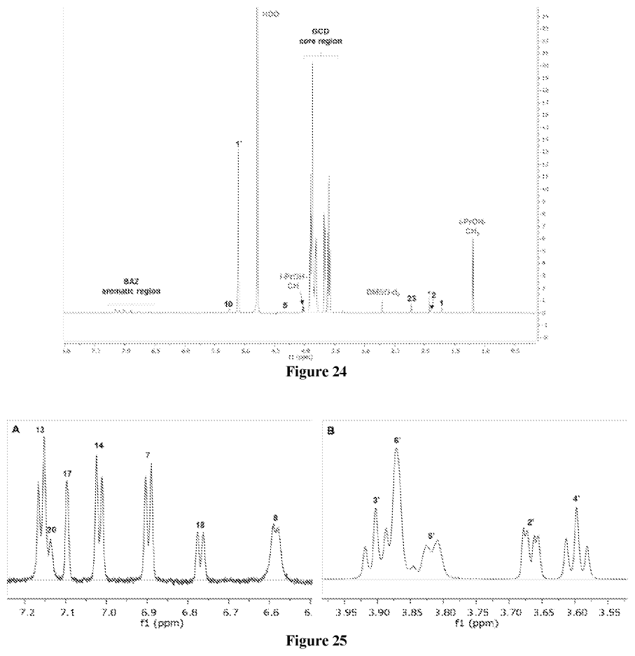

According to these findings, BAZ was able to penetrate to the cavity of GCD to form an inclusion complex with the proposed structure depicted in FIG. 23/A, as derived from 2D ROESY data analysis.

The simultaneous presence of ROESY cross-peaks between 3′ and 13/14 as well as 6′ and 13/14 suggested that a second type of BAZ:GCD complex could have formed with the proposed structure depicted in FIG. 23/B, where the B ring of BAZ penetrated the cavity of GCD, possibly through the primary ring. In order to compose more realistic picture of the proposed structures of these inclusion complexes, the 3D model of BAZ, sourced from PubChem database (https://pubchem.ncbi.nlm.nih.gov/compound/Bazedoxifene), was used to depict the complexes-see FIG. 23.

### Interaction Study of BAZ and GCD in the Prepared Complex

In this experiment, the pre-prepared complex of BAZ:GCD was dissolved in deuterated solvent and evidence for inclusion was investigated by employing NMR spectroscopy in the same manner as it was in the case of the model complex.

The ¹H NMR spectrum of the prepared BAZ:GCD complex was collected as shown in FIG. 24 with partial assignment. The expanded section of the BAZ aromatic region and GCD core region were observed as shown in FIG. 25A and FIG. 25B, respectively, with assignment of the peaks being determined.

Besides the previously observed interactions between the methyl protons of BAZ (23) and the inner protons of GCD (3′, 5′ and 6′), clear cross-peaks between the inter-aromatic methylene unit of BAZ (10) and the inner protons of GCD supported the existence of a structure akin to that depicted in FIG. 23A. Furthermore, interactions of this methylene unit of BAZ (10) with the external protons of GCD (2′ and 4′) were detected. It suggested formation of an external complex as well, though other moieties of BAZ involved in such interactions were not observed.

Summarizing these findings, host-guest complexes with the proposed structures depicted in FIG. 23 were supported by NMR spectroscopy studies of the prepared BAZ:GCD complex.

### Example 9: Antiviral Testing—Cytotoxicity and Mean Virus Titer

### Cytotoxicity

The toxicity of the test substance was measured in a culture of Vero-E6 cells (ATCC CRL-1586). Cells were cultured in a 96-well microtiter plate (2×10⁴ cells per well) for 24 h at 37° C. and 5% CO₂. Then, the test substance was added to the cells in a concentration range of 0-15.2 μg/μL, and the thus treated cells were subsequently incubated for another 48 h. The viability of the cells was then determined using the Cell Counting Kit-8 kit (Dojindo Molecular Technologies, Munich, Germany), exactly according to the manufacturer ‘s instructions.

### Mean Virus Titer

Confluent culture of Vero-E6 cells (ATCC CRL-1586) was used to test for antiviral activity. Cells were cultured in a 96-well microtiter plate (2×10⁴ cells per well) for 24 h at 37° C. and 5% CO₂. The culture medium was then aspirated and replaced with fresh medium containing the test substance in a concentration range of 0-3.8 μg/μL. At the same time, the cells were infected with the SARS-CoV-2 virus (strain SARS-CoV-2/human/Czech Republic/951/2020) at a multiplicity of infection of 0.1. The thus treated and infected cells were incubated for 48 h at 37° C. and 5% CO2. The medium was then aspirated from the culture wells and the virus titer was determined by plaque titration (Stefanik et al., Microorganisms 2021, 9 (3), 471).

TABLE 5
Antiviral effect

| | Antiviral effect [log reduction | Cytotoxicity |
|---|---|---|
| | of pfu/mL at the max non-toxic | (max. non-toxic |
| | concentration], Vero-E6 cells | concentration) |
| Formulation | (ATCC CRL-1586), EC₅₀ | [μM] |
| BAZ:SBECD | 4 log reduction at | >5 μM |
| | concentrations <10 μg/μL | |
| BAZ:GCD | 1 log reduction at | >5 μM |
| | concentrations <10 μg/μL | |

### Example 10: Preparation of Bazedoxifene Samples

### Lyophilized Product

0.6 g Bazedoxifene acetate and 7 g SBECD were dissolved in 80-120 g distilled water. The mixture of compounds was stirred at 500 rpm while heated to 44-60° C. on a heated magnetic stirrer for 120 min. After mixing, the solution was lyophilized to obtain a dry product, with 2-10% moisture. This lyophilized product was analyzed by DSC, mass spectrometry (MS) and NMR spectroscopy.

### Nebulizer Solution Concentrate

0.6 g Bazedoxifene acetate and 7 g SBECD were dissolved in 100 g distilled water. The mixture of compounds was stirred at 500 rpm with heating to 44-60° C. on a heated magnetic stirrer for 120 min. The final concentration of bazedoxifene in the product is 5 mg/mL. The product is stored inside a glass vial contained within a paper box in a refrigerator under monitored temperature (2-8° C., data logger).

### Example 11: Administration of Bazedoxifene to Rats

The study objective was to evaluate safety of the test formulation bazedoxifene acetate with SBECD after single intravenous (i.v.) and single intranasal (i.n.) administration to rats.

The study was conducted on 52 rats (26 males and 26 females). Animals were divided into two dose groups (G1 and G2) and two control groups (C1 and C2).

The G1 dose group contained 10 males and 10 females. Each rat received a single intravenous administration of 0.5 mg/kg of bazedoxifene acetate in SBECD. The G2 dose group contained 10 males and 10 females. Each rat received a single intranasal administration of 0.5 mg/kg of bazedoxifene acetate in SBECD. The C1 control group contained 3 males and 3 females. Each rat received a single intravenous administration of saline (NaCl 0.9%). The C2 control group contained 3 males and 3 females. Each rat received a single intranasal administration of saline (NaCl 0.9%).

During the acclimation and study period, the animals were fed a standard pellet diet Altromin (Germany) ad libitum. The quality of diet was monitored and the corresponding certificates was available in the Test facility IPHYS CAS archive.

Animals were housed under conventional laboratory conditions (Building HII, in room No. 022). The room temperature was 20-24° C., and the relative humidity of air was 30-70%. The room was monitored and ventilated. The lighting regime was 12 hours light and 12 hours dark. Feed and water containers were changed and sanitized at least twice a week. Safe (Germany) was used as bedding and was changed at least twice a week.

No clinical signs of toxicity were observed in the animals of both dose groups after single intravenous and intranasal administration of the bazedoxifene in the dose of 0.5 mg/kg under the test condition used. A single intravenous or intranasal administration of the bazedoxifene in the dose of 0.5 mg/kg did not cause pathological lesions in the lungs and trachea of rats.

No clinical signs of toxicity were observed in animals from the two control groups treated intravenously and intranasally with saline under the conditions used in this study.

All animals survived until their scheduled necropsy (Isoflurin anesthesia) 7 days after the administration (day D8) and received a gross necropsy. All gross pathological changes were recorded. Lung tissues (L and R caudal lobe) and trachea were retained for further histopathological examination.

### Example 12: A Phase I Clinical Trial for Bazedoxifene

Safety, tolerability and pharmacokinetics of novel inhalation form of bazedoxifene acetate (BAZE-X1) were evaluated in a Phase 1 Randomized, Double-Blinded, Placebo-Controlled, single dose study of BAZE-X1 in healthy volunteers. Sterile solution of bazedoxifene acetate with SBECD (sulfobutyl ether beta cyclodextrin) in water for injection (WFI) was used for preparation of concentrate. The concentrate is depicted in Table 6.

TABLE 6
BAZE-X1 formulation

| | | Quantity in | |
|---|---|---|---|
| | | 1 batch | |
| Starting | Quantity | (5,000 vials; | |
| Materials | in 1 mL | 32,000 ml) | Function |
| Bazedoxifene | 5.64 mg | 179.2 g | Active |
| acetate | (equivalent to 5 | | substance |
| | mg bazedoxifene) | | |
| SBECD | 67.7 mg | 2,166.4 g | Excipient |
| (sulfobutylated | | 2,307.0 g | solubilization |
| beta | | (humidity | substance |
| cyclodextrin) | | 6.1% RH) | |
| Water for | Up to 1 ml | Up to 32,000 ml | Solvent |
| Injection | | | |

In the first cohort (completed in September 2021), 8 healthy volunteers (6 with nebulizing formulation of bazedoxifene acetate at dose level of 1.13 mg and 2 with placebo) were enrolled and completed the study. Among those 8 subjects only 1 male was enrolled and treated with BAZE-X1. Mean age was 32.2 years in a group treated with BAZE-X1 and 33.5 years in a group receiving placebo.

After the analysis of the data of this cohort of 8 subjects, there were no safety issues observed. All safety assessments performed did not reveal any safety concern. No adverse events occurred during the course of study.

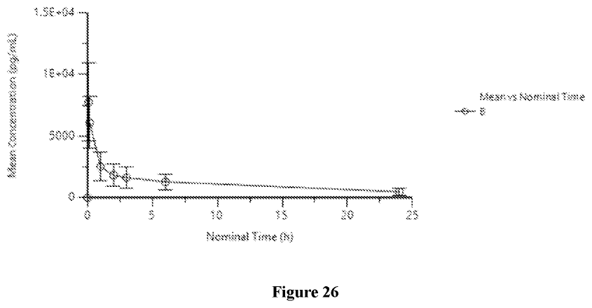

The analysis of pharmacokinetic data showed that observed plasma levels (pg/mL) and the AUC values (h·pg/mL) of bazedoxifene were very low, in majority near the limit of quantitation. The time to maximum plasma concentration was observed at the first measurement (after 10 min). The AUC curves in first 10-30 min sharply declined which predicts that drug is not retained in the lung tissue in healthy volunteers and it is absorbed in plasma very quickly. Elimination half-lives were lower (mean=21 h), and values of CL/F were slightly higher in comparison to oral and intravenous data, which indicated more rapid elimination after administration by inhalation. Plasma concentrations over time and pharmacokinetic parameters are depicted in FIG. 26 and Tables 7 and 8.

TABLE 7
Plasma concentrations

| | Nominal Time (h) | | | | | | | |
|---|---|---|---|---|---|---|---|---|
| | 0.00 | 0.08 | 0.17 | 1.00 | 2.00 | 3.00 | 6.00 | 24.00 |
| Subject | Concentration (pg/mL) | | | | | | | |
| N | 6 | 6 | 6 | 6 | 6 | 6 | 6 | 6 |
| Mean | 0.0 | 7752.8 | 6079.1 | 2524.1 | 1827.3 | 1633.0 | 1288.3 | 484.2 |
| SD | 0.0 | 3168.3 | 2105.4 | 1173.9 | 878.8 | 830.2 | 633.1 | 292.2 |
| CV % | x | 40.87 | 34.63 | 46.51 | 48.09 | 50.84 | 49.14 | 60.34 |
| Min | 0.0 | 4366.9 | 3578.5 | 1138.6 | 807.6 | 683.5 | 584.5 | 241.6 |
| Max | 0.0 | 13104.0 | 8918.4 | 4081.4 | 2967.3 | 2699.0 | 2110.1 | 1004.7 |
| Median | 0.0 | 6626.5 | 5317.6 | 2181.6 | 1593.1 | 1391.7 | 1122.5 | 401.9 |
| Geometric Mean | x | 7265.4 | 5783.5 | 2293.8 | 1648.1 | 1455.8 | 1157.4 | 422.3 |

Concentrations below LLOQ are presented as zero and were set to zero for descriptive statistics.

TABLE 8
Pharmacokinetic parameter estimates

| | AUClast | | Cmax | | | | | |
|---|---|---|---|---|---|---|---|---|
| Subject | (h*pg/mL [symbol] | AUCINF_obs | (pg/mL [symbol] | Tmax | Lambda_z | HL_Lambda_z | AUC_% Extrap_obs | Tlag |
| N | 6 | 6 | 6 | 6 | 6 | 6 | 6 | 6 |
| Mean | 28405.6 | 37553.8 | 7752.8 | 0.08 | 0.05890 | 12.32 | 23.11 | 0.00 |
| SD | 13804.7 | 19960.0 | 3168.3 | 0.01 | 0.01394 | 2.81 | 7.83 | 0.00 |
| CV % | 48.60 | 53.15 | 40.87 | 8.13 | 23.66 | 22.84 | 33.90 | x |
| Min | 13026.6 | 18348.6 | 4366.9 | 0.07 | 0.04502 | 8.87 | 13.68 | 0.00 |
| Max | 48192.5 | 70508.1 | 13104.0 | 0.08 | 0.07817 | 15.40 | 31.65 | 0.00 |
| Median | 25037.9 | 33733.7 | 6626.5 | 0.08 | 0.05653 | 12.51 | 24.17 | 0.00 |
| Geometri [symbol] | 25619.4 | 33462.6 | 7265.4 | 0.08 | 0.05755 | 12.04 | 21.93 | x |

[symbol]

indicates data missing or illegible when filed

### Example 13: A Phase II Clinical Study on the Safety AND Efficacy of Bazedoxifene and BECD

This is a Phase 2 study to evaluate safety and tolerability of bazedoxifene, concentrated solution for nebulization (BAZE-X1) in patients who were admitted to the hospital with moderate to severe COVID-19 pneumonia. The study will consist of 2 parts: Part A (Open-label, Non-randomized) and Part B (Randomized, Double-blind).

Duration of the treatment in both parts of the study (A and B) is minimum 3 (considered as 9 doses) and maximum 5 (considered as 15 doses) days according to the length of hospitalization and patient's condition at the discretion of the investigator. Booster dose (applicable only for Part B) is considered as equivalent to three 1.13 mg doses of bazedoxifene acetate or placebo.

### Part a (Open-Label, Non-Randomized)

Part A will enroll 10 patients in total and investigated IMP will be BAZE-X1. All 10 patients will be administered with one dose level of 1.13 mg of bazedoxifene acetate (equivalent to 1 mg bazedoxifene) three times daily according to dosage regimen described in the section 1.8.1.1.

### Part B (Randomized, Double-Blinded)

Part B will start with Cohort 1 and enroll 8 patients. Patients within each cohort will be randomized between active treatment (6 patients) and placebo (2 patients).

In Cohort 1, one dose level of 1.13 mg of bazedoxifene acetate (equivalent to 1 mg bazedoxifene) or placebo administered three times daily will be investigated. Single booster dose of 3.39 mg of bazedoxifene acetate or placebo on the Day 1 may be given to patients enrolled in Part B if it is not possible to administer 3 doses of 1.13 mg of bazedoxifene acetate or placebo on that day. On the Day 2, patient who received booster dose will continue with standard treatment schedule (3 doses of 1.13 mg of bazedoxifene acetate or placebo per day). IMP will be administered according to dosage regimen described in the section 1.8.1.2.

After completion of the Cohort 1, safety data will be evaluated by established DSMB. Further cohorts with respective dose escalation are planned to be involved in the trial. DSMB will take part in defining the escalated dose for the subsequent cohorts based on the interim analysis of results from the Cohort 1. Additional cohorts will be implemented in the study protocol via substantial amendment, after receiving approval from RA and EC.

### Primary Endpoints

The primary endpoints will be the safety and tolerability of single dose level of BAZE-X1 administered three times daily in patients with COVID-19 pneumonia measured by incidence and spectrum of all adverse events.

### Secondary Endpoints

The secondary endpoints include efficacy of BAZE-X1 on prevention of cytokine storm measured by change in leukocyte, lymphocyte, neutrophil count and plasma concentration of CRP, D/dimers, ferritin and IL-6 at EoT compared to baseline (D1); efficacy of antiviral effect of BAZE-X1 measured by change in concentration of N (nucleocapsid) antigen in blood at EoT compared to baseline (D1); efficacy of BAZE-X1 in patients with COVID-19 pneumonia measured by number of respiratory (invasive/non-invasive mechanical ventilation) and cardiovascular (infusion of vasopressor/inotrope at any dose) support free days within 28 days of study period, calculated from baseline (D1) to discharge or last follow-up (D28); efficacy of BAZE-X1 measured by number of days on supplementary oxygen therapy (face mask up to 15 L/min) within 28 days of study period, calculated from baseline (D1) to discharge or last follow-up (D28); efficacy of BAZE-X1 measured by number of days on HFNC (high-flow nasal cannula devices) within 28 days of study period, calculated from baseline (D1) to discharge or last follow-up (D28); efficacy of BAZE-X1 in patients administered with oxygen (supplementary oxygen therapy or HFNC) at the baseline (D1) measured by the change in oxygen flow (L/min) at EoT and D28 compared to baseline (D1) efficacy of BAZE-X1 measured by hospital discharge time within 28 days of study period, calculated from baseline (D1) to discharge or last follow-up (D28); hospital mortality at D28; and efficacy measured by changes in Clinical status of patient (using 7-point ordinal scale): at baseline (D1), EoT and D28: (1-Death; 2-Hospitalized, on invasive mechanical ventilation or ECMO; 3-Hospitalized, on non-invasive ventilation or high flow oxygen devices; 4-Hospitalized, requiring supplemental oxygen; 5-Hospitalized, not requiring supplemental oxygen/requiring ongoing medical care (COVID-19 related or otherwise); 6-Hospitalized, not requiring supplemental oxygen/no longer requires ongoing medical Care; 7-Not hospitalized).

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A composition of bazedoxifene or a pharmaceutically acceptable salt thereof and sulfobutyl ether beta-cyclodextrin.

2. A liquid pharmaceutical formulation comprising bazedoxifene or a pharmaceutically acceptable salt thereof and sulfobutyl ether beta-cyclodextrin, and further comprising at least one pharmaceutically acceptable solvent.

3. A solid pharmaceutical formulation comprising bazedoxifene or a pharmaceutically acceptable salt thereof and sulfobutyl ether beta-cyclodextrin, and further comprising at least one pharmaceutically acceptable excipient.

4. An inhalable pharmaceutical formulation comprising bazedoxifene or a pharmaceutically acceptable salt thereof and sulfobutyl ether beta-cyclodextrin, and further comprising at least one pharmaceutically acceptable solvent and at least one pharmaceutically acceptable propellant and/or aerosol-forming gas.

5. The pharmaceutical formulation of any one of claims 2 to 4, wherein bazedoxifene and sulfobutyl ether beta-cyclodextrin are in a molar ratio of at least about 1:1.

6. The pharmaceutical formulation of claim 5, wherein bazedoxifene and sulfobutyl ether beta-cyclodextrin are in a molar ratio of at least about 1:5.

7. A method of treatment of viral infections caused by coronaviruses, comprising the step of administering the composition of claim 1 or the pharmaceutical formulation of claims 2 to 6 or a pharmaceutically acceptable salt thereof and sulfobutyl ether beta-cyclodextrin (in a therapeutically effective dose) to a subject in need of such treatment.

8. The method according to claim 7, wherein the composition is administered by inhalation, intranasally, orally, or intraocularly.

9. The method according to claim 7 or 8, wherein the composition is administered by means of a nebulizer, a nasal spray, an oral formulation, or eye drops.

10. The method according to claim 7, wherein the composition is administered intravenously.

11. A method of treatment of infections caused by the virus SARS-CoV-2, comprising the step of administering the composition of claim 1 or the pharmaceutical formulation of claims 2 to 6 to a subject in need of such treatment.

12. The method according to claim 11, wherein the composition is administered by inhalation, intranasally, orally, or intraocularly.

13. The method according to claim 11 or 12, wherein the composition is administered by means of a nebulizer, a nasal spray, an oral formulation, or eye drops.

14. The method according to claim 11, wherein the composition is administered intravenously.

15. A method of treatment of cancer, comprising the step of administering the pharmaceutical formulation of claims 2 to 6 (in a therapeutically effective dose) to a subject in need of such treatment.

16. The method according to claim 15, wherein the pharmaceutical formulation is administered by inhalation, intranasally, orally, or intraocularly.

17. The method according to claim 15 or 16, wherein the pharmaceutical formulation is administered by means of a nebulizer, a nasal spray, a liquid formulation, or eye drops.

18. The method according to claim 15, wherein the pharmaceutical formulation is administered intravenously.

19. The method according to claim 15, wherein the cancer is a breast cancer or a pancreatic cancer.

20. A method of treatment of osteoporosis, comprising the step of administering the pharmaceutical composition of claims 2 to 6 (in a therapeutically effective dose) to a subject in need of such treatment.

21. The method according to claim 20, wherein the osteoporosis is postmenopausal osteoporosis.

22. A salt of bazedoxifene with sulfobutyl ether beta-cyclodextrin.

23. A composition according to any one of claims 1 to 6 for use as a medicament.

24. A composition according to any one of claims 1 to 6 for use in the treatment of a disease selected from viral infections caused by coronaviruses, SARS-CoV-2 infection, cancer and osteoporosis.
